(19) United States
(12) Patent Application Publication
(10) Pub. No.: US 2019/0161879 A1
(43) Pub. Date: May 30, 2019

(54) MANUFACTURING METHOD OF MICROPOROUS FILTER FOR AEROSOL GENERATING NEBULIZER AND MICROPOROUS FILTER BY USING THEREOF

(71) Applicant: DONGGUK UNIVERSITY GYEONGJU CAMPUS INDUSTRY ACADEMY COOPERATION FOUNDATION, Gyeongju-si, Gyeongsangbuk-do (KR)

(72) Inventors: Ki Chang NAM, Yangcheon-gu, Seoul (KR); Hyo Chul JI, Nonsan-si, Chungcheongnam-do (KR); Jae Jong KIM, Hwaseong-si, Gyeonggi-do (KR)

(73) Assignee: DONGGUK UNIVERSITY GYEONGJU CAMPUS INDUSTRY ACADEMY COOPERATION FOUNDATION, Gyeongju-si, Gyeongsangbuk-do (KR)

(21) Appl. No.: 16/313,614

(22) PCT Filed: Jun. 23, 2017
(86) PCT No.: PCT/KR2017/006640

§ 371 Date: Dec. 27, 2018

(30) Foreign Application Priority Data
Jun. 29, 2016 (KR) 10-2016-0082023
May 26, 2017 (KR) 10-2017-0065416

(51) Int. Cl. C25D 3/56; C25D 3/50; A61M 11/00; B01D 39/14

(52) U.S. Cl. CPC C25D 3/567; B01D 39/14; A61M 11/001; C25D 3/50

(57) ABSTRACT

The present invention relates to a method for manufacturing a porous filter for fine spraying and a porous filter manufactured by the method. A nickel-palladium alloy material porous filter manufactured according to the present invention exhibits excellent corrosion resistance and metal ion elution mitigating effects and can adjust the size of drug particles, thereby allowing a drug to arrive at the desired site. In addition, a plating liquid having a particular composition in the present invention allows the manufacture of a porous filter with a desired thickness by effectively lowering the stress of palladium (Pd), and thus, in a fine sprayer for treating a respiratory disease, a microporous filter, which is capable of delivering a drug to the vicinity of alveoli while effectively preventing the elution of metal elements due to a drug, apparatus vibration, and the like, and a fine sprayer, which uses the microporous filter, can be manufactured.

### CROSS REFERENCE TO RELATED APPLICATIONS

This application claims benefit under 35 U.S.C. § 371 of PCT Application No. PCT/KR2017/006640 filed Jun. 23, 2017, the entire contents of which are incorporated herein by reference.

### BACKGROUND OF THE DISCLOSURE

### 1. Field of the Disclosure

The present invention relates to a method for manufacturing a porous filter for fine spraying, and more specifically to a method for manufacturing a porous filter for fine spraying which uses nickel-palladium (Ni—Pd) alloy plating solution to secure durability and biological safety and a porous filter manufactured using thereof.

### 2. Description of Related Art

Recently, a nebulizer come to the market and is widely used to deliver drugs to the target area effectively in treating respiratory diseases such as bronchial asthma and chronic obstructive pulmonary disease (COPD). More specifically, the nebulizer is a porous membrane based fine spraying generating device that can spray aerosol effectively to deliver disease-specific drugs in the intake under normal breathing conditions, and maximize the effect of drug delivery. Meanwhile, when using the device as described above, the delivery of the drug to the lungs is influenced by the particle size of the drug and inhalation container. In particular, the ideal particle size of the drug is 1 μm to 5 μm to be delivered around the alveoli. To control the particles of the drug, it is important to develop a new porous filter.

In the past, the Electroforming Method is mostly used as a plating method for manufacturing porous filters. The method is also called the ‘electroplating method’, and is a method for making or duplicating metal products by electrodeposition. More specifically, it is a method for making or duplicating metal products by exfoliating an electrodeposited layer from a substrate after electrodeposition a metal in a certain thickness on the substrate having flat or engraved/or embossed parts by electrolysis of the metal salt solution. In this plating process, nickel (Ni) is mostly used as an electroplating metal due to its high-gloss and resistance to corrosion. However, when the nickel contacts with salt water, sweat, and cosmetics, it may cause a problem of releasing toxic nickel ions by chemical reaction. For this reason, despite the potentiality for medical use, it is necessary to develop new technologies to prevent the elution of toxic nickel ions.

Therefore, the development of methods and devices to manufacture a porous filter which is made of fine spray type metal (e.g., alloy) having excellent resistance to corrosion to use medical purpose is an important task, and some researches are being conducted (JP patent publication No. 2005-296737), but they are lacking.

### SUMMARY OF THE DISCLOSURE

The present invention has been made in order to solve the above problems, and the present inventors have found excellent resistance to corrosion of the porous filter manufactured by an electroplating method using a nickel-palladium (Ni—Pd) plating solution having a composition within a specific range, and the present invention has been completed based on the above.

Accordingly, it is an object of the present invention to provide a method for manufacturing the porous filter, which includes the following steps: (a) preparing a negative electrode plate for electroforming in which a pattern is formed; (b) immersing the negative electrode plate prepared in step (a) in a porous filter-plating solution containing 20% by weight to 80% by weight of nickel and 15% by weight to 80% by weight of palladium, and then forming a plating film by applying a current thereto; and (c) exfoliating the plating film formed in step (b) from the negative electrode plate.

Further, it is another object of the present invention to provide a porous filter manufactured by the method as described above.

Furthermore, it is another object of the present invention to provide a fine spraying device including the porous filter as described above.

However, the technical problem to be solved by the present invention is not limited by the problems as described above, and other issues not mentioned can be clearly understood by those skilled in the art from the following description.

In order to achieve the objects as described above, the present invention is to provide a method for manufacturing a porous filter, the method including (a) preparing a negative electrode plate for electroforming in which a pattern is formed; (b) immersing the negative electrode plate prepared in step (a) in a porous filter-plating solution containing 20% by weight to 80% by weight of nickel and 15% by weight to 80% by weight of palladium, and then forming a plating film by applying a current thereto; and (c) exfoliating the plating film formed in step (b) from the negative electrode plate.

In one embodiment of the present invention, step (b) can be performed by immersing the negative electrode plate prepared in step (a) in the porous filter-plating solution containing 27% by weight to 60% by weight of nickel and 40% by weight to 73% by weight of palladium, and then forming a plating film by applying a current thereto.

In one embodiment of the present invention, step (b) can be performed under the condition of a temperature of the plating solution of 35° C. to 65° C.

In one embodiment of the present invention, step (b) can be performed under the condition of the applied current of 0.05 A to 15 A.

In one embodiment of the present invention, step (b) can be performed under the condition of a plating time of 0.5 minutes to 65 minutes.

In one embodiment of the present invention, step (b) can be performed under the condition of the temperature of the plating solution of 39° C. to 48° C.

In one embodiment of the present invention, step (b) can be performed under the condition of the applied current of 0.5 A to 4.5 A.

In one embodiment of the present invention, step (b) can be performed under the condition of a plating time of 40 minutes to 65 minutes.

In one embodiment of the present invention, the porous filter-plating solution in step (b) can include diamine palladium dichloride (Pd(NH3)2Cl2) and nickel sulfamate tetrahydrate (Ni(NH2SO3)24H2O).

In one embodiment of the present invention, the porous filter-plating solution in step (b) can further include nickel chloride (NiCl2).

In one embodiment of the present invention, the porous filter-plating solution in step (b) can further include 1% by weight to 20% by weight of a first brightener.

In one embodiment of the present invention, the porous filter-plating solution in step (b) can further include 1% by weight to 20% by weight of a second brightener.

In one embodiment of the present invention, the porous filter-plating solution in step (b) can further include 1% by weight to 20% by weight of a buffer.

In one embodiment of the present invention, the porous filter-plating solution in step (b) can further include 1% by weight to 20% by weight of a surfactant.

In one embodiment of the present invention, the first brightener can be tannic acid (C28H22O11).

In one embodiment of the present invention, the second brightener can be 1,4-butanediol (OH(CH2)4OH).

In one embodiment of the present invention, the buffer can be boric acid (H3BO3).

In one embodiment of the present invention, the surfactant can be sodium lauryl sulfate.

The present invention provides a porous filter manufactured by the method as described above.

In one embodiment of the present invention, the porous filter can have a thickness of 14 μm to 60 μm.

In one embodiment of the present invention, the porous filter can have a plurality of pores.

In one embodiment of the present invention, the pores can have a diameter of 0.5 μm to 5 μm.

In one embodiment of the present invention, the pores can have a diameter of 1 μm to 5 μm.

The present invention provides a fine spraying device including the porous filter as described above.

According to the present invention, when a porous filter of nickel-palladium alloy materials which is manufactured by electroplating using a nickel-palladium (Ni—Pd) plating solution having a composition within a specific range is used as a fine spraying filter, it is confirmed that the filter has excellent durability, thereby the elution of metal elements from the filter is reduced due to external factors such as corrosion and vibration energy.

Further, according to the present invention, it is possible to effectively reduce the stress of palladium (Pd) through the plating solution having a specific composition to produce a porous filter having a desired thickness, and the size of the drug particle is adjusted due to the pores formed in the porous filter so that drugs can be reached out to the deepest part of the body's lung.

Accordingly, the porous filter according to the present invention is applicable to a fine spraying device for treating respiratory diseases which effectively prevents the elution of metal elements due to vibration of drugs and devices and effectively transfers drugs to the periphery of alveoli.

### BRIEF DESCRIPTION OF THE DRAWINGS

FIG. 1 illustrates the results of measuring the pore size and thickness of a porous filter manufactured by proceeding with plating under the conditions of an applied current of 2 A and a plating time of 10 minutes.

FIG. 2 illustrates the results of measuring the pore size and thickness of a porous filter manufactured by proceeding with plating under the conditions of an applied current of 1.5 A and a plating time of 20 minutes.

FIG. 3 illustrates the results of measuring the pore size and thickness of a porous filter manufactured by proceeding with plating under the conditions of an applied current of 1.5 A and a plating time of 90 minutes.

FIG. 4 illustrates the results of measuring the pore size and thickness of a porous filter manufactured by proceeding with plating under the conditions of an applied current of 1.5 A and a plating time of 13 minutes and additionally plating under the conditions of an applied current of 2.5 A and a plating time of 32 minutes.

FIG. 5 illustrates a result of measuring the diameter of the porous filter manufactured under the optimum plating conditions according to an embodiment of the present invention.

FIG. 6 is a schematic diagram of a porous filter according to an embodiment of the present invention.

FIG. 7 is a schematic diagram of an experiment for biological safety inspection of a porous filter according to an embodiment of the present invention.

FIG. 8 illustrates the result of confirming the biological safety of the nickel-plated porous filter.

FIG. 9 illustrates the results of confirming the biological safety of a nickel-palladium porous filter manufactured according to an embodiment of the present invention.

### DETAILED DESCRIPTION

Hereinafter, the present invention is described in detail.

The present invention provides a method for manufacturing the porous filter, which includes the following steps: (a) preparing a negative electrode plate for electroforming in which a pattern is formed; (b) immersing the negative electrode plate prepared in step (a) in a porous filter-plating solution containing 20% by weight to 80% by weight of nickel and 15% by weight to 80% by weight of palladium, and then forming a plating film by applying a current thereto; and (c) exfoliating the plating film formed in step (b) from the negative electrode plate.

In the present invention, step (a) is a step of preparing a negative electrode plate for electroplating. At this embodiment, the surface of the negative electrode plate may have various fine patterns thereon according to the shape of a plating film to be obtained as described above. A method such as a lithography or imprint is preferably used in order to form fine patterns. The lithography method may be more preferably used but is not limited thereto.

Meanwhile, in the drug delivery using the fine spraying device, the size of the drug particle is critical in delivering the drug to the target site. More specifically, when the size of the drug particle is 5 μm or more, it is mostly deposited in the throat portion of the oral cavity. When the size of the drug particle is 1 μm to 5 μm, the drug particle is transferred from the airway to the peripheral bronchus. When the size of the drug particle is 1 μm or less, the drug can be transferred to the periphery of the alveoli. In this case, the size of drug obtained by the fine spraying device may be adjusted by the porous filter. Accordingly, in order to effectively deliver the drug to the periphery of the alveoli, the pore diameter of the porous filter is preferably 1 μm to 5 μm.

Therefore, when forming the pattern on the negative electrode plate using the lithography method, it is preferable to form a pattern against the size of the desired pores of the porous filter. More specifically, a photoresist is spin-coated on the surface of the negative electrode plate, and the plate is placed on a hot plate having 100° C. for drying for 1 minute. Thereafter, a finely patterned photomask having the desired pore size, which has been already prepared is placed on the photoresist, and the photoresist is melted to form a pattern on the negative electrode plate according to a fine pattern using lithography. Next, the patterned negative electrode plate is printed in a developing solution, placed on a hot plate at 100° C. for drying for 2 minutes.

Meanwhile, in the case of a porous membrane made of a metal material (for example, nickel (Ni)) which is applied to a conventional fine spraying device, elution of constituent metal elements occurs due to corrosion and vibration energy due to long-term use, thereby it was difficult to be used for medical purposes. To this end, the present invention includes a step of plating the plate with the nickel and palladium alloy to prevent elution of the constituent metal elements. This can effectively prevent the elution of metal components that may harm the human body.

More specifically, step (b) of the present invention is a process that the patterned negative electrode plate prepared in step (a) is immersed in a porous filter-plating solution having a composition within a specific range, and then a current is applied thereto to form a plating film. At this time, the porous filter-plating solution may preferably include 20% by weight to 80% by weight of nickel and 15% by weight to 80% by weight of palladium, more preferably 27% by weight to 60% by weight of nickel and 40% by weight to 73% by weight of palladium, most preferably 40% by weight of nickel and 60% by weight of palladium, but is not limited thereto.

Meanwhile, the porous filter-plating solution may include diamine palladium dichloride (Pd(NH3)2Cl2) and nickel sulfamate tetrahydrate (Ni(NH2SO3)24H2O) as the main element, and may further include nickel chloride (NiCl2). It may additionally include additives necessary for plating.

For example, the additive may include a first brightener, a second brightener, a buffer and/or a surfactant.

In this case, the porous filter-plating solution may have additives including 1% by weight to 20% by weight of the first brightener, 1% by weight to 20% by weight of the second brightener, 1% by weight to 20% by weight of the buffer, and 1% by weight to 20% by weight of the surfactant.

In this case, tannic acid (C28H22O11) is preferably used as the first brightener, 1,4-butanediol (OH(CH2)40H) is preferably used as the second brightener, boric acid (H3BO3) is preferably used as the buffer, and sodium lauryl sulfate as the surfactant, but the present invention is not limited thereto.

Further, in adding the brightener to the porous filter-plating solution, it is preferable to start the plating with the addition ratio of the first brightener and the second brightener set at 2:1, and then to perform the plating in a ratio of 1:3 to 4.

Next, the porous filter-plating solution is put into a plating bath. The patterned negative electrode plate is immersed, and then a current is applied to perform electroforming.

In this case, plating conditions for the electroforming may preferably have a plating solution temperature of 35° C. to 65° C., an applied current of 0.05 A to 15 A and a plating time of 0.5 minutes to 65 minutes, more preferably a plating solution temperature of 35° C. to 60° C., an applied current of 0.1 A to 10 A and a plating time of 20 minutes to 65 minutes, furthermore preferably a plating solution temperature of 35° C. to 55° C., an applied current of 0.15 A to 5 A and a plating time of 30 minutes to 65 minutes, and most preferably a plating solution temperature of 39° C. to 48° C., an applied current of 0.5 A to 4.5 A and a plating time of 40 minutes to 65 minutes. However, the plating conditions can be changed depending on the desired plating thickness and pore size without limitation.

Meanwhile, it is important to remove the internal stress in order to form the desired thickness of the plating in the electroforming plating. Conventionally, any plating may have internal stress, which may generate stress due to the type of plating, the composition of the plating solution, the kind of additive, and the like. Such stress affects the adhesion and the like, thereby promoting the exfoliation of the plating film. For example, in the case of a plating film formed by plating to form a thin film, the internal stress may be small. However, in the case of a plating film formed by plating to form a thick film, the stress gradually increases, thereby causing other problems such as deformation and exfoliation.

Further, palladium (Pd) contained in the porous filter-plating solution used in an embodiment of the present invention has high stress of the metal itself. Thus, as the plating is performed, deformation, exfoliation and the like occur before obtaining the desired thickness of the plating film, which makes plating difficult. In order to solve such a problem, an embodiment of the present invention provides a nickel-palladium (Ni—Pd) alloy plating solution having a composition ratio within a specific range, thereby lowering the stress of palladium (Pd). Thus, plating may proceed without deformation, exfoliation and other problems until the desired thickness of the plating film is obtained. At this time, the thickness of the plating film formed in step (b) of the present invention may preferably be 14 μm to 60 μm, more preferably 30 μm to 40 μm, and most preferably 35 μm to 40 μm. This makes it possible to obtain a plating film having a thickness with the desired durability (such as tensile strength, hardness, and elastic modulus).

In the present invention, step (c) is a process of exfoliating the plating film formed in step (b) from the negative electrode plate. In this case, the exfoliated plating film has pores and is made of a nickel-palladium alloy, thereby enhancing the durability and corrosion resistance thereof. Thus, the porous filter may have high stability and control the size of the drug particle. Further, in step (c), a chemical treatment may be performed on the surface using various releasing agents such as oxides, hydroxides, and metal salts in order to separate the plating film from the negative electrode plate without damaging the plating film. Accordingly, the surface adhesive strength may be reduced to smoothly peel off the plating film.

Further, as another aspect of the present invention, the present invention provides a porous filter formed by the manufacturing method as described above.

In yet another aspect of the present invention, the present invention provides a fine spraying device including the porous filter as described above.

Hereinafter, preferred Examples of the present invention is described in order to facilitate understanding of the present invention. However, the following Examples are provided only for the purpose of easier understanding of the present invention, and the present invention is not limited by the following Examples.

### Example 1. Selection of Optimum Conditions for the Production of the Porous Filter According to the Present Invention

Experiments were conducted to produce a porous filter having an optimum thickness and pore size according to the present invention as follows.

Specifically, it was to confirm whether the desired thickness and pore size were obtained while varying the plating temperature, the applied current and the plating time. The plating conditions are shown in Table 1 below.

TABLE 1

| | Temp | | Current | Time | Total | pore size | thickness |
|---|---|---|---|---|---|---|---|
| No | (° C.) | pH | (A) | (min) | Time (min) | Outlet (μm) | (μm) |
| 1 | 27 | 6.7 | 2 | 10 | 10 | 40~43 | 9.0 |
| 2 | 27 | 6.7 | 1.5 | 20 | 20 | 34~50 | 10 |
| 3 | 40 | 7.1 | 1.5 | 90 | 90 | 9~10 | 39~41 |
| 4 | 40 | 7.1 | 1.5 | 13 | 45 | 23 | 35~40 |
| | | | 2.5 | 32 | | | |

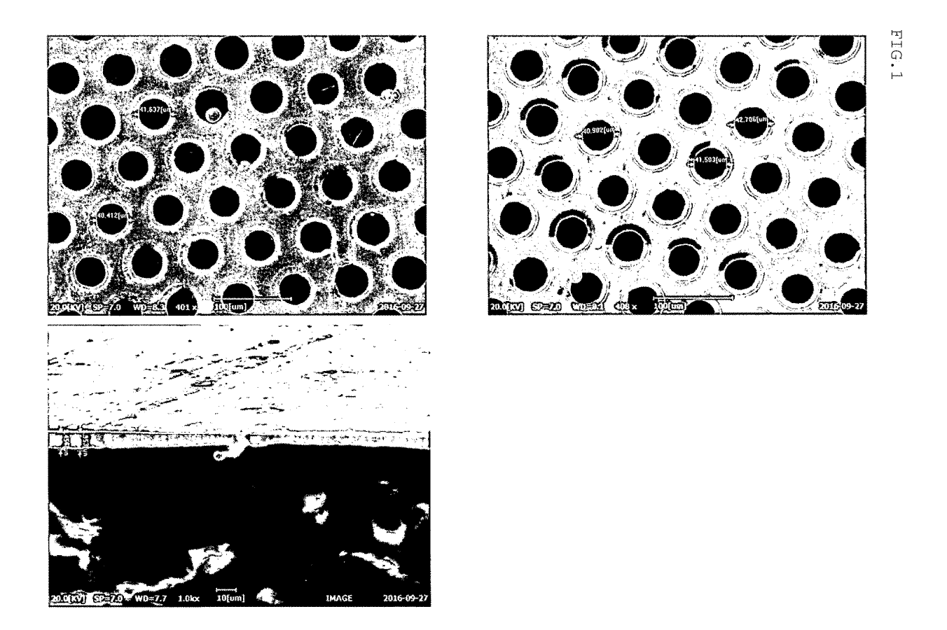

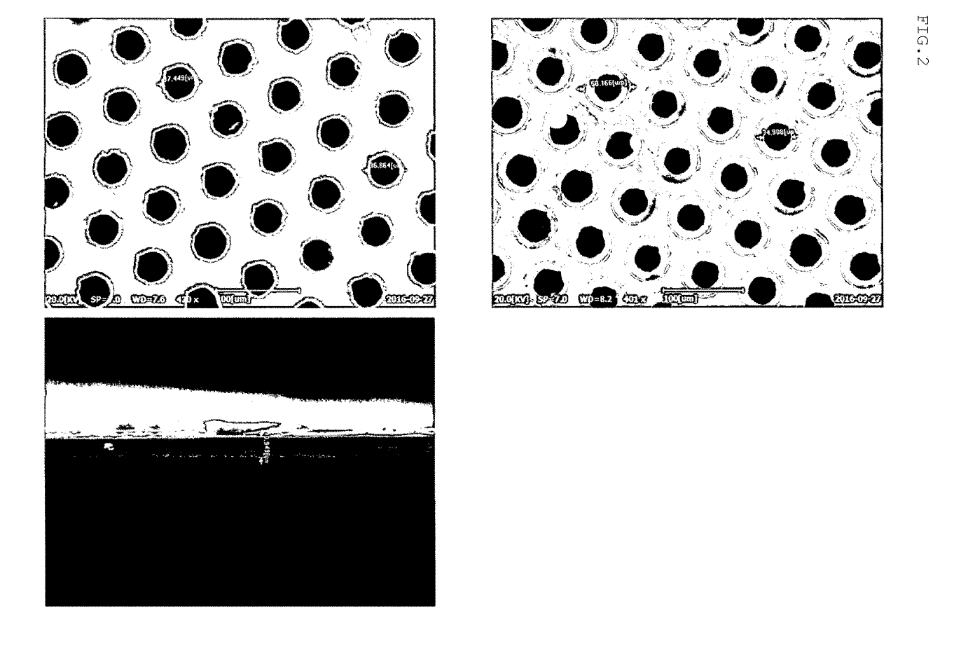

More specifically, plating was performed at the plating temperature of 27° C. which is somewhat low, the applied current of 2 A and the plating time of 10 minutes which is short. As illustrated in FIG. 1, the pore sizes thereof were 40 μm to 43 μm, and the thickness thereof was 9.0 μm, indicating that the thickness of plating film was thin, and the pore size was large. Next, plating was performed at the plating temperature of 27° C. which is somewhat low, the applied current of 1.5 A and the plating time of 20 minutes. As illustrated in FIG. 2, the pore sizes thereof were 34 μm to 50 μm, and the thickness thereof was 10 μm, indicating that the thickness of plating film was thin, and the pore size was large, which is the same as the results illustrated in FIG. 1.

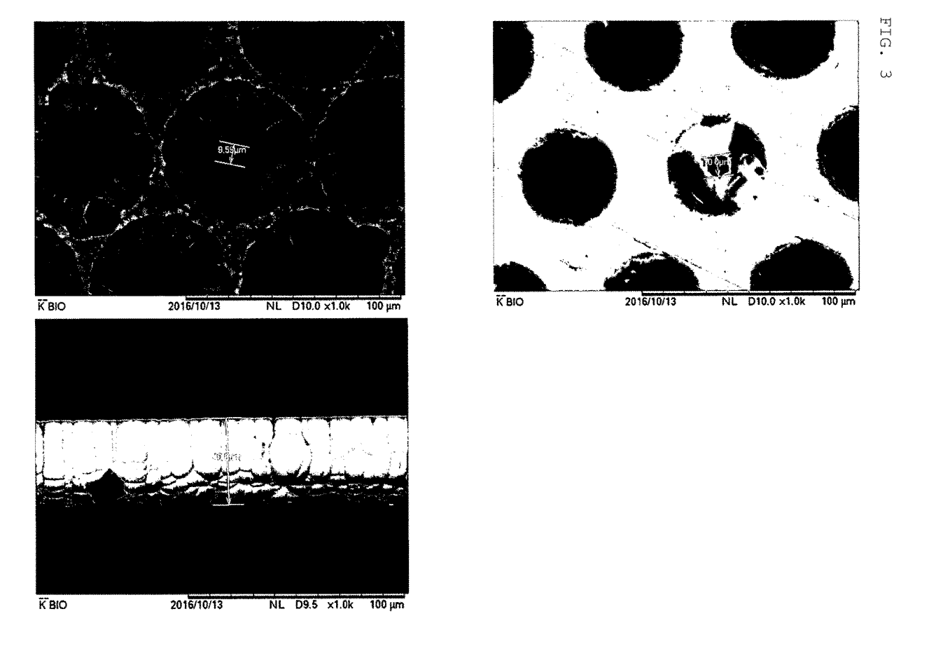

Thus, the present inventors have changed plating conditions to form the desired thickness. Plating was conducted under the conditions of a plating temperature of 40° C., the applied current of 1.5 A and the plating time of 90 minutes. As results illustrated in FIG. 3, the pore size was 9 μm to 10 μm, and the thickness was 39 μm to 41 μm, indicating that the desired thickness was obtained, but the desired pore size was not obtained.

Next, in order to shorten the plating time, the plating was performed by increasing the current intensity. Specifically, the plating was performed at a plating temperature of 40° C. and an applied current of 1.5 A for 13 minutes, and further plating was performed at 2.5 A for 32 minutes.

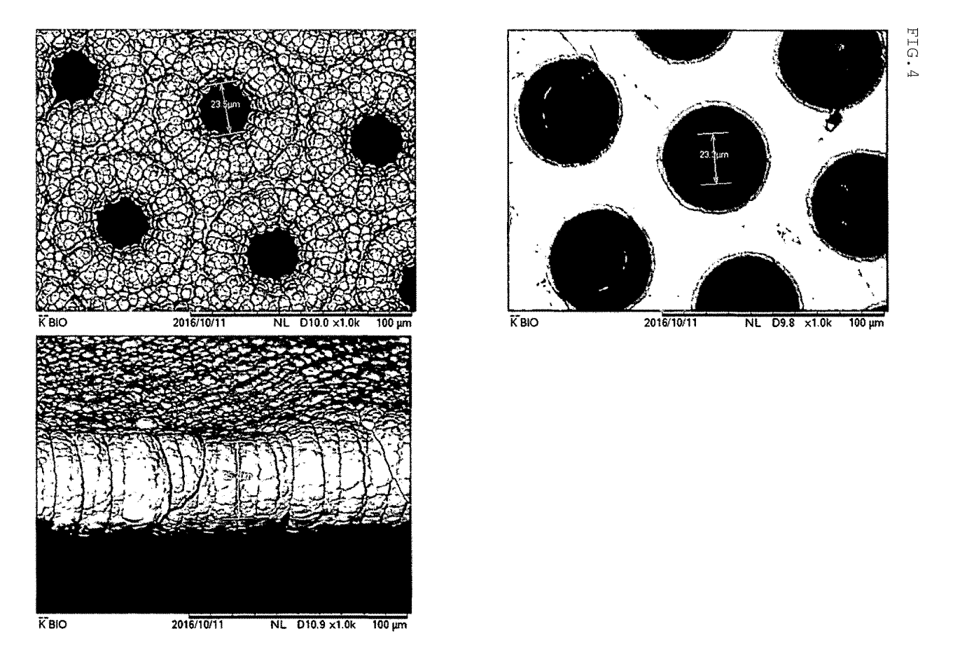

As results illustrated in FIG. 4, the pore size was 23 μm, and the thickness was 35 μm to 40 μm, indicating that the target thickness was obtained, but the pore size was still not reduced. Thus, it was confirmed that the target thickness and the pore size could be formed by adjusting the current condition over time among the plating conditions.

According to the results of the study, experiments were conducted to select the plating conditions for obtaining optimal thickness and pore size in which the plating temperature was 39° C. to 48° C., the applied current was 0.5 A to 4.5 A, and the plating time was 40 minutes to 65 minutes, and the ratio of palladium and nickel contained in the plating solution was varied.

TABLE 2

| | | | | | | | | Ni |
|---|---|---|---|---|---|---|---|---|
| | Temp | Current | Time | Total | pore size | thickness | Pd Weight | Weight |
| No | (° C.) | (A) | (min) | Time (min) | Outlet (μm) | (μm) | (%) | (%) |
| 1 | 41.2 | 1.0 | 15 | 40 | 5.0 | 14.7 | 71.3 | 28.7 |
| | | 3.0 | 20 | | | | | |
| | | 0.5 | 5 | | | | | |
| 2 | 41.4 | 4.5 | 40 | 44 | 11.6 | 39.1 | 40.9 | 59.1 |
| | | 2.0 | 3 | | | | | |
| | | 0.7 | 1 | | | | | |
| 3 | 41.4 | 4.5 | 40 | 44 | 13.2 | 36.0 | 59.6 | 40.4 |
| | | 2.0 | 3 | | | | | |
| | | 0.7 | 1 | | | | | |
| 4 | 47.1 | 1.0 | 15 | 45 | 7.4 | 15.0 | 72.8 | 27.2 |
| | | 3.0 | 25 | | | | | |
| | | 0.5 | 5 | | | | | |
| 5 | 39.8 | 4.5 | 45 | 52 | 3.4 | 56.5 | 53.9 | 46.1 |
| | | 2.0 | 5 | | | | | |
| | | 0.7 | 2 | | | | | |
| 6 | 39.7 | 4.5 | 45 | 52 | 1.0 | 42.2 | 63.1 | 36.9 |
| | | 2.0 | 5 | | | | | |
| | | 0.7 | 2 | | | | | |
| 7 | 40.5 | 4.5 | 40 | 62 | 2.9 | 43.3 | 63.6 | 36.4 |
| | | 3.0 | 10 | | | | | |
| | | 1.5 | 10 | | | | | |
| | | 0.5 | 2 | | | | | |

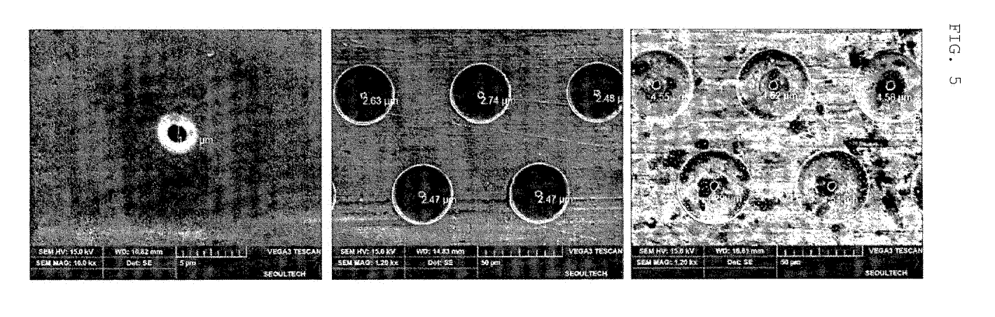

The porous filter was manufactured according to the conditions shown in Table 2. It was confirmed that the pore size was adjusted to 1 μm to 5 μm while the thickness was 14 μm to 60 μm (See FIG. 5).

Meanwhile, the operation of the fine spraying device induces the spraying of the liquid through the vibration energy generated by the vibration element. In this case, the vibration energy may make the porous filter to be cracked or destroyed. Thus, in order to prevent the porous filter from being damaged by the vibration energy, the higher the hardness, the more advantageous it is.

Accordingly, in the present invention, experiments were conducted to check Vickers hardness according to the weight ratio of palladium and nickel in the plating solution. In this case, the Vickers hardness measurement method for measuring the Vickers hardness is a standard method for measuring the hardness of very hard surface material. In this method, the surface is measured in terms of length and time with reference to a reference pressure using a pyramidal diamond, and the size engraved from the pyramidal diamond indentor was calculated to obtain hardness. Accordingly, experiments were conducted to examine the Vickers hardness of the porous filter according to the weight ratio of palladium and nickel contained in the plating solution according to the method as described above. The results are shown in Table 3 below.

TABLE 3

| | Pd (weight); Ni (weight) | | Vickers hardness |
|---|---|---|---|
| Electroplating Condition | (%) | | (HV_{0.2}) |
| 0.5~4.5 A, 65 min | 64.0 | 36.0 | 532.5 |
| 0.5~3.0 A, 45 min | 72.8 | 27.2 | 417.7 |
| 0.7~4.5 A, 44 min | 45.3 | 54.7 | 472.2 |

As shown in Table 3, it was confirmed that when the ratio of the palladium and nickel weight percent of the plating solution was 64:36, the Vickers hardness thereof was the highest.

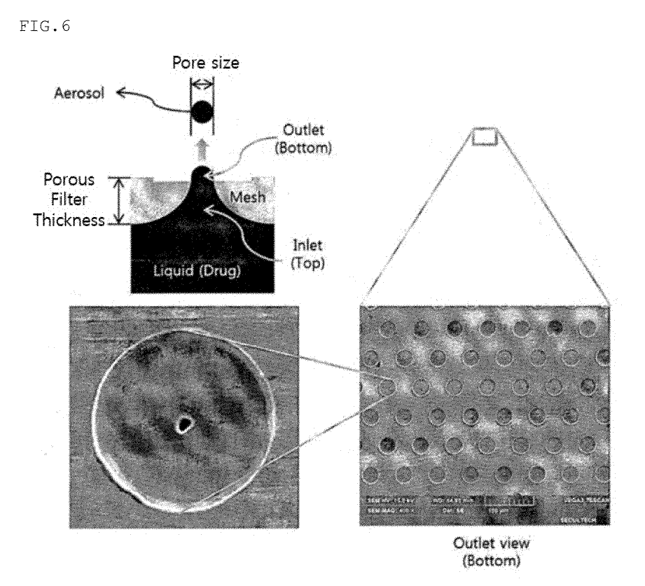

According to the above results, optimum conditions for manufacturing a porous filter having a desired thickness and pore size were confirmed, and the porous filter thus manufactured is illustrated in FIG. 6.

### Example 2. Confirmation of Elution of Toxic Substances by Fine Spraying

As described above, the porous filter according to the present invention was provided by plating of nickel and palladium alloy in order to effectively prevent the elution of the metal component, and the biological safety evaluation test was conducted for confirming the effect of preventing the elution of the metal component. The biological safety evaluation test was performed by direct diffusion method among the ISO 10993-5 Tests in vitro cytotoxicity, which is an international standard.

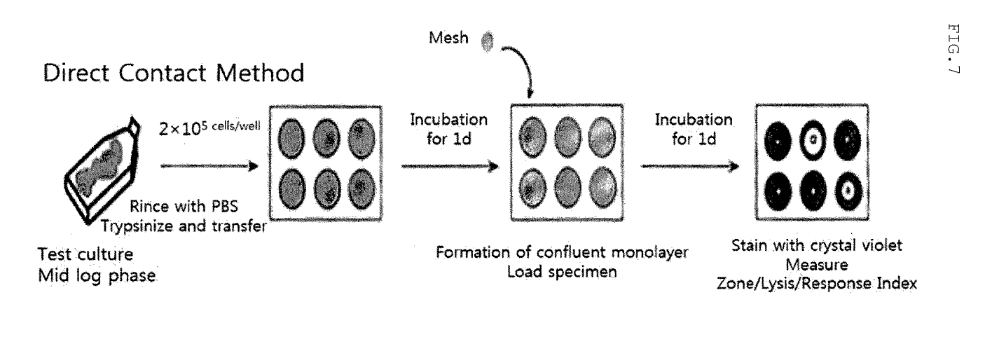

Specifically, the cell line used for the cytotoxicity test was L-929 (fibroblast), and GFP-transfected L-929 cells were used for imaging. The porous filter was washed and sterilized before the experiment and then placed on the surface of the cells previously cultured for 24 hours. Thus, the cytotoxicity of the substance released from the porous membrane was evaluated. In this case, a schematic diagram of the cytotoxicity experiment is illustrated in FIG. 7.

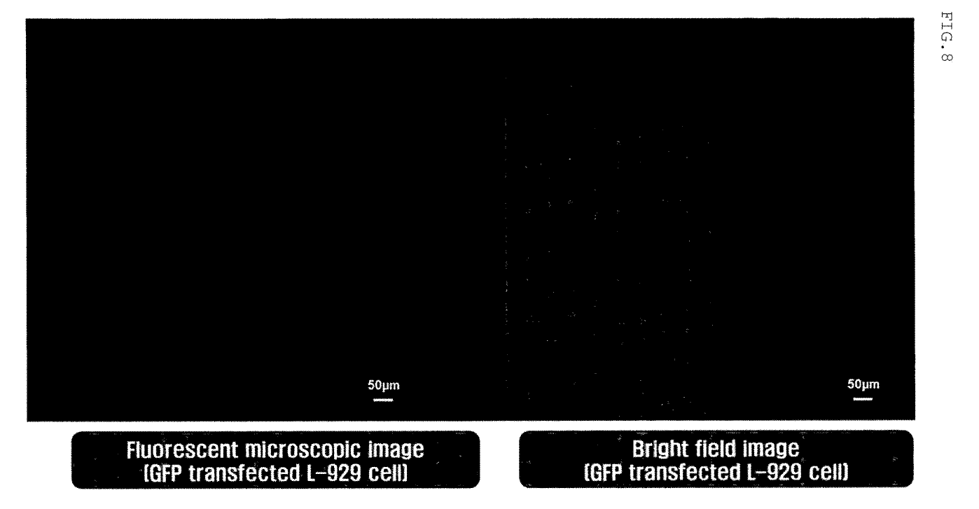

As results illustrated in FIG. 8, in the case of the nickel-plated porous filter, the evaluation of the cytotoxicity of the substance released from the porous filter (24 h) confirmed that all cells died due to toxicity released from the nickel-plated porous filter.

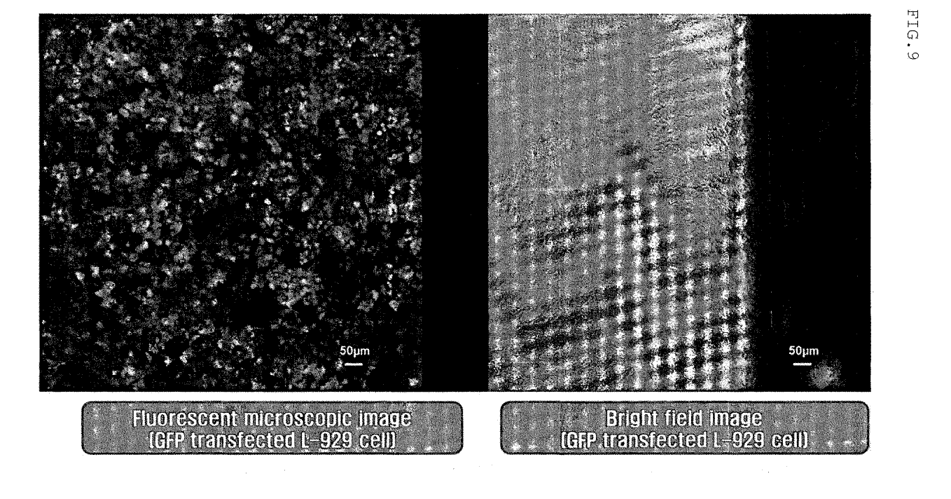

On the other hand, as illustrated in FIG. 9, in the case of the porous filter plated with the nickel and palladium according to the present invention, evaluation of the cytotoxicity of the substance released from the porous filter (24 h) specifically confirmed that all of the cells were observed to be alive, thereby indicating no cytotoxicity, unlike the nickel-plated porous filter.

It is understood by those skilled in the art that the above description of the present invention is intended to be illustrative, and various changes may easily be made in another specific formation without changing the technical ideas or essential features of the present invention. It is, therefore, to be understood that the above-described embodiments are illustrative in all aspects and not restrictive.

According to the present invention, it is possible to effectively reduce the stress of palladium (Pd) through the plating solution having a specific composition to produce a porous filter having a desired thickness. The size of the drug particle is adjusted due to the pores formed in the porous filter so that drugs can reach out the deepest part of the body's lung. Accordingly, the porous filter according to the present invention is applicable to a fine spraying device for treating respiratory diseases which effectively prevents the elution of metal elements due to vibration of drugs and devices and effectively transfers drugs to the periphery of alveoli.

1. A method for manufacturing a porous filter, the method comprising the following steps:
(a) preparing a negative electrode plate for electroforming in which a pattern is formed;
(b) immersing the negative electrode plate prepared in step (a) in a porous filter-plating solution containing 20% by weight to 80% by weight of nickel and 15% by weight to 80% by weight of palladium, and then forming a plating film by applying a current thereto; and
(c) exfoliating the plating film formed in step (b) from the negative electrode plate.

2. The method of claim 1, wherein step (b) is performed by immersing the negative electrode plate prepared in step (a) in the porous filter-plating solution containing 27% by weight to 60% by weight of nickel and 40% by weight to 73% by weight of palladium, and then forming a plating film by applying a current thereto.

3. The method of claim 1, wherein step (b) is performed with a plating solution temperature of 35° C. to 65° C.

4. The method of claim 1, wherein step (b) is performed with an applied current of 0.05 A to 15 A.

5. The method of claim 1, wherein step (b) is performed with a plating time of 0.5 minutes to 65 minutes.

6. The method of claim 1, wherein step (b) is performed with a plating solution temperature of 39° C. to 48° C.

7. The method of claim 1, wherein step (b) is performed with an applied current of 0.5 A to 4.5 A.

8. The method of claim 1, wherein step (b) is performed with a plating time of 40 minutes to 65 minutes.

9. The method of claim 1, wherein the porous filter-plating solution in step (b) includes diamine palladium dichloride (Pd(NH3)2Cl2) and nickel sulfamate tetrahydrate (Ni(NH2SO3)24H2O).

10. The method of claim 9, wherein the porous filter-plating solution in step (b) further includes nickel chloride (NiCl2).

11. The method of claim 1, wherein the porous filter-plating solution in step (b) further includes 1% by weight to 20% by weight of a first brightener.

12. The method of claim 11, wherein the porous filter-plating solution in step (b) further includes 1% by weight to 20% by weight of a second brightener.

13. The method of claim 11, wherein the porous filter-plating solution in step (b) further includes 1% by weight to 20% by weight of a buffer.

14. The method of claim 11, wherein the porous filter-plating solution in step (b) further includes 1% by weight to 20% by weight of a surfactant.

15. The method of claim 11, wherein the first brightener is tannic acid (C28H22O11).

16. The method of claim 12, wherein the second brightener is 1,4-butanediol (OH(CH2)4OH).

17. The method of claim 13, wherein the buffer is boric acid (H3BO3).

18. The method of claim 14, wherein the surfactant is sodium lauryl sulfate.

19. A porous filter produced by the method of claim 1.

20. The porous filter of claim 19, wherein the porous filter has a thickness of 14 μm to 60 μm.

21. The porous filter of claim 19, wherein the porous filter has a plurality of pores.

22. The porous filter of claim 21, wherein the pores have a diameter of 0.5 μm to 5 μm.

23. The porous filter of claim 21, wherein the pores have a diameter of 1 μm to 5 μm.

24. A fine spraying device comprising the porous filter according to claim 19.
